(19) United States
(12) Patent Application Publication
(10) Pub. No.: US 2025/0174321 A1
(43) Pub. Date: May 29, 2025

(54) TECHNIQUES FOR TRANSMITTING UNSTRUCTURED DATA BETWEEN DIFFERENT SYSTEMS

(71) Applicant: Flatiron Health, Inc., New York, NY (US)

(72) Inventors: Mariel Boyd Canale, Bronxville, NY (US); Dylan Russell Meacham, Tokyo (JP); Tyler Steven Stodden, Brooklyn, NY (US); Thomas Howard Marlette, Brooklyn, NY (US); Ursula Anne Alwang, Monterey, CA (US)

(73) Assignee: Flatiron Health, Inc., New York, NY (US)

(21) Appl. No.: 18/924,751

(22) Filed: Oct. 23, 2024

Related U.S. Application Data
(60) Provisional application No. 63/603,296, filed on Nov. 28, 2023.

(51) Int. Cl. G16H 10/60; G16H 15/00; H04L 9/40

(52) U.S. Cl. CPC G16H 10/60; G16H 15/00; H04L 63/083

(57) ABSTRACT

Described herein are techniques for ingesting data from multiple source systems that store data in conformance with a common specification. The techniques generate queries for the source systems based on the common specification and receive datasets in response to the queries. Described herein are also techniques of transmitting data from a source system into data records of a destination system in which data records have a structure different from that of source system data records. Data in the source system may be unstructured.

### RELATED APPLICATIONS

This application claims priority under 35 U.S.C. § 119(e) to U.S. Provisional Application Ser. No. 63/603,296, entitled “TECHNIQUES FOR TRANSMITTING UNSTRUCTURED DATA BETWEEN DIFFERENT SYSTEMS”, filed Nov. 28, 2023, which is hereby incorporated by reference in its entirety.

### FIELD

Described herein are techniques for transmitting data between systems. For example, the techniques may be used to ingest data from electronic health record (EHR) systems into a data abstraction system. As another example, the techniques may be used to transmit abstracted data from a data abstraction system into data records of an electronic data capture (EDC) system.

### BACKGROUND

An electronic health record (EHR) may store data associated with patients. Data may be added into the EHR by clinicians (e.g., physicians and/or nurses) during the course of treatment. The data may include structured data (e.g., values stored in fields specified by a structure) and unstructured data. Unstructured data may include documents (e.g., clinical visit note (CVN) documents) storing notes from clinicians. The EHR may store a record of all documents associated with a patient. The documents may include information about the patient such as medical history, diagnosis of medical conditions, status of medical conditions, and/or other information. Certain information about patients that may be useful for clinical studies (e.g., as part of pharmaceutical development and testing) needs to be abstracted from the documents.

Documents may be analyzed by subject matter experts in order to derive information about subjects. For example, a subject matter expert may review CVN documents associated with a cancer patient to determine a status of the patient's cancer, a prognosis of the cancer, a response of the patient to treatment, and/or other information about the patient. Information abstracted from CVN documents may be stored in datasets that are used for clinical studies.

### SUMMARY

Described herein are techniques for ingesting data from multiple different source systems (e.g., EHR systems). The source systems may be configured to transfer data according to a common specification (e.g., the FHIR specification developed by HL7). The techniques leverage the common specification to employ a query generation process that is uniform across all the source systems. The techniques allow retrieval of data from the source systems without requiring integration of a destination system (e.g., an abstraction system) with custom interfaces of the source systems.

Described herein are also techniques for transmitting data (e.g. unstructured data) from a source system (e.g., an abstraction system) to a destination system (e.g., an electronic data capture (EDC) system). The source system and the destination system may have data records with disparate data structures. For example, data in the source system may not adhere to a particular structure while data records in the destination system may adhere to a particular data structure definition. The techniques: (1) locate fields in destination system data records to which to transmit source system field values; and (2) format the values for storage in the fields of the destination system data records.

Some embodiments provide a system for ingesting data from a plurality of source systems into a destination system, the plurality of source systems storing the data in conformance with a common specification indicating a standard for transmitting datasets, the plurality of source systems including a first source system and a second source system. The system comprises: a processor configured to execute a plurality of modules, the plurality of modules comprising: a query generation module configured to request data from the plurality of source systems, the query generation module configured to: generate a first query requesting a first dataset stored in the first source system, the first query including a first value of at least one field designated by the common specification for referencing a dataset; generate a second query requesting a second dataset stored in the second source system, the second query including a second value of the at least one field designated by the common specification for referencing a dataset; a communication module configured to communicate, through a communication network, with the plurality of source systems, the communication module configured to: transmit, through the communication network, the first query to the first source system and the second query to the second source system; and receive, through the communication network, after transmission of the first query and the second query: the first dataset from the first source system; and the second dataset from the second source system; and a storage module configured to store, in a datastore accessible by the destination system, the first dataset and the second dataset.

In some embodiments, the common specification is the Fast Healthcare Interoperability Resources (FHIR) specification.

In some embodiments, each of at least some of the plurality of source systems is an electronic health record (EHR) system storing clinical records for a plurality of subjects.

In some embodiments, the at least one field designated by the specification for uniquely referencing the dataset comprises a subject identifier that uniquely identifies a subject that the dataset is associated with in a particular source system. In some embodiments, the system further comprises memory storing, for each of a plurality of subjects, respective values of the subject identifier used by the plurality of source systems to identify the subject, wherein: the query generation module is configured to determine the first value of the at least one field and the second value of the at least one field by performing: identify, in the memory, a value of the subject identifier used by the first source system to uniquely reference a first subject, wherein the first dataset is associated with the first subject in the first source system; and identify, in the memory, a value of the subject identifier used by the second source system to uniquely reference the first subject, wherein the second dataset is associated with the first subject in the second source system.

In some embodiments, the first dataset comprises a first set of unstructured data stored in the first source system and the second dataset comprises a second set of unstructured data stored in the second source system. In some embodiments, the first set of unstructured data comprises a first set of clinical visit note (CVN) documents associated with a first subject and the second set of unstructured data comprises a second set of CVN documents associated with a second subject.

In some embodiments, the processor is further configured to execute a subject identification module configured to: identify, from among a plurality of subjects, a subject with a destination system identifier for the subject that is associated with an identifier for the subject used by the first source system; and trigger generation of the first query to request data from the first source system after identifying the subject.

In some embodiments, the system further comprises memory storing sets of authentication credentials for the plurality of source systems and the communication module is further configured to: authenticate the system to the first source system using a first one of the sets of authentication credentials for the first source system to obtain a first access token; authenticate the system to the second source system using a second one of the sets of authentication credentials for the second source system to obtain a second access token; and wherein the communication module is configured to transmit, through the communication network, the first query to the first source system and the second query to the second source system by performing: transmit, through the communication network, the first query to the first source system using the first access token; transmit, through the communication network, the second query to the second source system using the second access token.

In some embodiments: the first dataset is associated with a first subject in the first source system; the second dataset is associated with the first subject in the second source system; determining the first value of the at least one field comprises determining a value of the at least one field used by the first source system to uniquely identify the first subject in the first source system; and determining the second value of the at least one field comprises determining a value of the at least one field used by the second source system to uniquely identify the first subject in the second source system.

In some embodiments: the first dataset is associated with a first one of the plurality of subjects; the second dataset is associated with a second one of the plurality of subjects; determining the first value of the at least one field comprises determining a value of the at least one field used by the first source system to uniquely identify data associated with the first subject in the first source system; and determining the second value of the at least one field comprises determining a value of the at least one field used by the second source system to uniquely identify data associated with the second subject in the second source system.

In some embodiments, the storage module is configured to store, in the datastore, the first dataset and the second dataset by performing: read the first dataset from the first source system into a cloud-based datastore, separate from the datastore of the system; read the second dataset from the second source system into the cloud-based datastore; and read the first dataset and the second dataset from the cloud-based datastore into the datastore of the destination system.

In some embodiments, the query generation module is configured to periodically request data associated with a plurality of subjects from the plurality of source systems.

Some embodiments provide method for ingesting data from a plurality of source systems into a destination system, the plurality of source systems storing the data in conformance with a common specification indicating a standard for transmitting datasets, the plurality of source systems including a first source system and a second source system. The method comprises using a processor to perform: generating a first query requesting a first dataset stored in the first source system, the first query including a first value of at least one field designated by the common specification for referencing a dataset; generating a second query requesting a second dataset stored in the second source system, the second query including a second value of the at least one field designated by the common specification for referencing a dataset; transmitting, through the communication network, the first query to the first source system and the second query to the second source system; receiving, through the communication network, after transmission of the first query and the second query: the first dataset from the first source system; and the second dataset from the second source system; and storing, in a datastore accessible by the destination system, the first dataset and the second dataset.

In some embodiments: the at least one field designated by the specification for uniquely referencing the dataset comprises a subject identifier that uniquely identifies a subject that the dataset is associated with in a particular source system; determining the first value of the at least one field and the second value of the at least one field comprises: identifying a value of the subject identifier used by the first source system to uniquely reference a first subject, wherein the first dataset is associated with the first subject in the first source system; and identifying a value of the subject identifier used by the second source system to uniquely reference the first subject, wherein the second dataset is associated with the first subject in the second source system.

In some embodiments, the method further comprises: identifying, from among a plurality of subjects, a subject with a destination system identifier for the subject that is associated with an identifier for the subject used by the first source system; and triggering generation of the first query to request data from the first source system after identifying the subject.

In some embodiments, the datastore stores sets of authentication credentials for the plurality of source systems, and the method further comprises: authenticating the system to the first source system using a first one of the sets of authentication credentials for the first source system to obtain a first access token; authenticating the system to the second source system using a second one of the sets of authentication credentials for the second source system to obtain a second access token; and wherein transmitting, through the communication network, the first query to the first source system and the second query to the second source system comprises: transmitting, through the communication network, the first query to the first source system using the first access token; transmitting, through the communication network, the second query to the second source system using the second access token.

In some embodiments: the first dataset is associated with a first subject in the first source system; the second dataset is associated with the first subject in the first source system; determining the first value of the at least one field comprises determining a value of the at least one field used by the first source system to uniquely identify the first subject in the first source system; and determining the second value of the at least one field comprises determining a value of the at least one field used by the second source system to uniquely identify the first user in the second source system.

In some embodiments: the first dataset is associated with a first one of the plurality of subjects; the second dataset is associated with a second one of the plurality of subjects; determining the first value of the at least one field comprises determining a value of the at least one field used by the first source system to uniquely identify data associated with the first subject in the first source system; and determining the second value of the at least one field comprises determining a value of the at least one field used by the second source system to uniquely identify data associated with the second subject in the second source system.

Some embodiments provide a non-transitory computer-readable storage medium storing instructions that, when executed by a processor, cause the processor to perform a method for ingesting data from a plurality of source systems into a destination system, the plurality of source systems storing the data in conformance with a common specification indicating a standard for transmitting datasets, the plurality of source systems including a first source system and a second source system. The method comprises: generating a first query requesting a first dataset stored in the first source system, the first query including a first value of at least one field designated by the common specification for referencing a dataset; generating a second query requesting a second dataset stored in the second source system, the second query including a second value of the at least one field designated by the common specification for referencing a dataset; transmitting, through the communication network, the first query to the first source system and the second query to the second source system; receiving, through the communication network, after transmission of the first query and the second query: the first dataset from the first source system; and the second dataset from the second source system; and storing, in a datastore accessible by the destination system, the first dataset and the second dataset.

Some embodiments provide a data transmission system for transmitting data from a source system into data records of a destination system. The system comprises: a processor; and a non-transitory computer-readable storage medium storing instructions that, when executed by the processor, cause the processor to: access, from the destination system, a data structure definition for the data records of the destination system, the data structure definition indicating: a plurality of destination system fields for storing values in the data records; and translation information for at least one of the plurality of destination system fields, the translation information comprising information for translating data obtained from the source system for storage in the at least one destination system field; transmit, using the data structure definition, data from a source system data record to a destination system data record having a structure defined by the data structure definition, the transmitting comprising: identify, using a field map that associates source system fields with respective ones of the plurality of destination system fields, at least one source system field from which to read a value; access a value of the at least one source system field from the source system data record; translate, using the translation information for the at least one destination system field in the data structure definition, the value of the at least one source system field to obtain a translated value; and store the translated value in the at least one destination system field of the destination system data record.

In some embodiments, the data from the at least one source system field is in a format different from a target format of the at least one destination system field, and translating, using the translation information for the at least one destination system field, the value of the at least one source system field to obtain the translated value comprises: formatting the value stored in the at least one source system field into the target format of the at least one destination system field.

In some embodiments, translating, using the translation information for the at least one destination system field, the value stored in the at least one destination system field to obtain the translated value comprises: converting the data into a structured representation for the at least one destination system field.

In some embodiments, the data structure definition includes a container that includes the plurality of fields. In some embodiments: the field map associates the source system data record with the container of the data structure definition; and the transmitting comprises: identifying, in the field map, the container of the data structure definition associated with the source system data record; accessing, from the data structure definition, the plurality of fields in the container; and identifying, using the field map, the at least one source system field associated with the at least one destination system field after accessing the plurality of fields in the container.

In some embodiments: the translation information for the field comprises: a plurality of candidate values of the at least one source system field; a plurality of translated values corresponding to the plurality of candidate values; and translating, using the translation information for the at least one destination system field, the value of the at least one source system field to obtain the translated value comprises: identifying one of the plurality of candidate values that is stored in the at least one source system field; and selecting the corresponding one of the plurality of translated values as the translated value for the at least one destination system field.

In some embodiments, storing the translated value in the at least one destination system field of a respective one of the data records associated with the subject comprises: generating an operational data model (ODM) extensive markup language (XML) file including the translated value; and transmitting the ODM XML file to the destination system for storage.

In some embodiments, the at least one source system field stores data abstracted from clinical visit note (CVN) documents associated with subjects. In some embodiments, the source system is an abstraction system and the destination system is an electronic data capture (EDC) system. In some embodiments, the source system data record is an SQLite file.

In some embodiments, transmitting the data from the source system data record to the destination system data record comprises transmitting unstructured data from the source system data record to the destination system data record.

Some embodiments provide a method for transmitting data from a source system into data records of a destination system. The method comprises using a processor to perform: accessing, from the destination system, a data structure definition for the data records of the destination system, the data structure definition indicating: a plurality of destination system fields for storing values in the data records; and translation information for at least one of the plurality of destination system fields, the translation information comprising information for translating data obtained from the source system for storage in the at least one destination system field; transmitting, using the data structure definition, data from a source system data record to a destination system data record having a structure defined by the data structure definition, the transmitting comprising: identifying, using a field map that associates source system fields with respective ones of the plurality of destination system fields, at least one source system field associated with the at least one destination system field; accessing a value of the at least one source system field from the source system data record; translating, using the translation information for the at least one destination system field in the data structure definition, the value of the at least one source system field to obtain a translated value; and storing the translated value in the at least one destination system field of the destination system data record.

In some embodiments, the data from the at least one source system field is in a format different from a target format of the at least one destination system field, and translating, using the translation information for the at least one destination system field, the value of the at least one source system field to obtain the translated value comprises: formatting the value stored in the at least one source system field into the target format of the at least one destination system field.

In some embodiments, the value stored in the at least one source system field comprises unstructured data, and translating, using the translation information for the at least one destination system field, the value stored in the at least one destination system field to obtain the translated value comprises: converting the unstructured data into a structured representation for the at least one destination system field.

In some embodiments: the data structure definition includes a container that includes the plurality of fields; the field map associates the source system data record with the container of the data structure definition; and the transmitting comprises: identifying, in the field map, the container of the data structure definition associated with the source system data record; accessing, from the data structure definition, the plurality of fields in the container; and identifying, using the field map, the at least one source system field associated with the at least one destination system field after accessing the plurality of fields in the container.

In some embodiments: the translation information for the field comprises: a plurality of candidate values of the at least one source system field; a plurality of translated values corresponding to the plurality of candidate values; and translating, using the translation information for the at least one destination system field, the value of the at least one source system field to obtain the translated value comprises: identifying one of the plurality of candidate values that is stored in the at least one source system field; and selecting the corresponding one of the plurality of translated values as the translated value for the at least one destination system field.

In some embodiments, storing the translated value in the at least one destination system field of a respective one of the data records associated with the subject comprises: generating an operational data model (ODM) extensive markup language (XML) file including the translated value; and transmitting the ODM XML file to the destination system for storage.

In some embodiments, transmitting the data from the source system data record to the destination system data record comprises transmitting unstructured data from the source system data record to the destination system data record.

Some embodiments provide a non-transitory computer-readable storage medium storing instructions that, when executed by a processor, cause the processor to perform a method for transmitting data from a source system into data records of a destination system. The method comprises: accessing, from the destination system, a data structure definition for the data records of the destination system, the data structure definition indicating: a plurality of destination system fields for storing values in the data records; and translation information for at least one of the plurality of destination system fields, the translation information comprising information for translating data obtained from the source system for storage in the at least one destination system field; transmitting, using the data structure definition, data from a source system data record to a destination system data record having a structure defined by the data structure definition, the transmitting comprising: identifying, using a field map that associates source system fields with respective ones of the plurality of destination system fields, at least one source system field associated with the at least one destination system field; accessing a value of the at least one source system field from the source system data record; translating, using the translation information for the at least one destination system field in the data structure definition, the value of the at least one source system field to obtain a translated value; and storing the translated value in the at least one destination system field of the destination system data record.

In some embodiments, the data from the at least one source system field is in a format different from a target format of the at least one destination system field, and translating, using the translation information for the at least one destination system field, the value of the at least one source system field to obtain the translated value comprises: formatting the value stored in the at least one source system field into the target format of the at least one destination system field.

The foregoing summary is non-limiting.

### BRIEF DESCRIPTION OF DRAWINGS

Various aspects and embodiments will be described with reference to the following figures. It should be appreciated that the figures are not necessarily drawn to scale. Items appearing in multiple figures are indicated by the same or a similar reference number in all the figures in which they appear.

FIG. 1A is an example environment in which some embodiments of the technology described herein may be implemented.

FIG. 1B illustrates an example implementation of the data transmission system of FIG. 1A for use in ingesting data from EHR systems into an abstraction system, according to some embodiments of the technology described herein.

FIG. 2 illustrates operation of a data transmission system for obtaining datasets from source systems, according to some embodiments of the technology described herein.

FIG. 3 shows an example of generating queries using subject identifiers stored in a datastore, according to some embodiments of the technology described herein.

FIGS. 4A-4B show an example response to a query generated by a data transmission system, according to some embodiments of the technology described herein.

FIG. 5 is an example process 500 for ingesting data into a destination system from multiple different source systems, according to some embodiments of the technology described herein.

FIG. 6 shows an example environment in which a data transmission system may operate, according to some embodiments of the technology described herein.

FIG. 7 is a diagram of an example source data record, according to some embodiments of the technology described herein.

FIG. 8 is a diagram of an example data structure definition, according to some embodiments of the technology described herein.

FIG. 9 is an example field map, according to some embodiments of the technology described herein.

FIG. 10 illustrates transmission of data by the data transmission system from a source data record to a destination data record, according to some embodiments of the technology described herein.

FIG. 11 is an example implementation of some embodiments of the technology described herein for transmitting data between EHR systems, an abstraction system, and an electronic data capture (EDC) system.

FIG. 12 is an example process for transmitting from a source system to a destination system, according to some embodiments of the technology described herein.

FIG. 13 is a diagram of an illustrative computing system that may be used in implementing some embodiments of the technology described herein.

### DETAILED DESCRIPTION

Described herein are techniques for efficiently ingesting data from multiple different source systems. The techniques leverage a common specification employed by the source systems to efficiently access data from the different systems. For example, the techniques may be used for ingesting unstructured data (e.g., clinical visit note (CVN) documents from different electronic health record (EHR) systems. Described herein are also techniques of transmitting data from a source system to a destination system for storage in a target data structure of the destination system. For example, the techniques may be employed for transmitting unstructured data (e.g., information abstracted from CVN documents) from a source system to a structured collection of data in a destination system.

The inventors have recognized that conventional techniques for accessing information stored in an EHR system by a system external to the EHR system are inefficient. To allow an external system to access data about subjects from an EHR system, conventional techniques require a customized integration of the external system with the EHR system. For example, the EHR system may have a proprietary application programming interface (API) and the external system would have to be integrated with the EHR system's API. This process could take months, if not one or more years, to complete. Moreover, if a system needs to access data from multiple different EHR systems, the system would need to be integrated with each of those EHR systems separately. Each time an EHR system needs to be integrated with the external system, additional functionality and configuration has to be performed in the external system to allow it to communicate with the EHR system.

The inventors have recognized and appreciated that many EHR systems store data in accordance with a common specification that indicates a standard according to which data can be transferred. The specification may include rules that may require data to be collected in a particular way and may specify one or more fields (e.g., metadata fields) that store information about sets of data. For example, one specification that may be used by EHR systems to store data associated with subjects (e.g., patients) is the Fast Healthcare Interoperability Resources (FHIR) specification published by HL7. The inventors recognized that the common specification according to which data is transferred in the EHR systems can be utilized to ingest data without requiring custom integrations with the EHR systems.

Accordingly, the inventors developed techniques that allow a system to leverage a common specification used by source systems (e.g., EHR systems) to access data from the source systems. The techniques allow the system to ingest data from the source systems without having to be integrated with a custom API of each of the source systems. The techniques thus eliminate the need for the development of functionality in a system for integration with each source system. Rather, the system implements a common query framework across all source systems that employ the specification. For example, the system may use the same process to generate queries for all the different source systems. The system is thus independent and not limited by proprietary interfaces (e.g., APIs) of the source systems that may vary across the source systems.

Some embodiments allow a system to request data from a given source system as soon as the system is given credentials for accessing the source system. The system may authenticate itself to the source system using the credentials. Once the system is authenticated, the system may begin issuing queries for data from the source system (e.g., by generating queries that leverage field(s) indicated by a specification used by the source system to reference data). For example, the system may use a particular field indicated by the FHIR specification as identifying a subject in an EHR system to request data associated with the subject from the EHR system.

Some embodiments allow a system to efficiently access unstructured data from source systems. Unstructured data may be stored in one or more files (e.g., documents) and may not be organized into fields of defined database structure. For example, an EHR system may store unstructured data such as physician notes from subjects' clinical visits in documents (referred to herein as “clinical visit note (CVN) documents”). The notes may be free-form notes written and/or dictated by physicians and stored in CVN documents. A specification (e.g., FHIR) may specify rules for identifying the unstructured data. For example, the FHIR specification indicates a particular resource (i.e., “DocumentReference”) that references a collection of data (e.g., CVN documents) associated with a subject. Some embodiments may leverage the particular resource to access unstructured data (e.g., CVN documents) from different EHR systems.

One challenge in transmitting data from a source system to a destination system is that data records of a source system may not store data in a structure used by the destination system to store data. For example, data in the source system may be unstructured. This prevents the destination system from automatically reading data from the source system into data records of the destination system. As an illustrative example, the source system may store data abstracted from CVN documents associated with multiple different subjects. The data may be abstracted by human experts and stored in various fields of data records in the source system. The abstracted data may need to be transmitted to an electronic data capture system (EDC) that stores data associated with subjects according to a particular structure for use in clinical study.

The inventors have recognized that conventional techniques of data transmission fail to efficiently transmit unstructured data from a source system (e.g., an abstraction system) to a destination system (e.g., an EDC system). Conventional techniques are unable to automatically transmit unstructured data from fields of the source system to the destination system such that the data adheres to a structure in which data is stored by the data records.

To address the above-described shortcomings of conventional techniques, the inventors have developed techniques that allow data to be transmitted from a source system into structured data records of a destination system. For example, the techniques allow the transmission of abstracted data from fields of an abstraction system to fields in structured data records of an EDC system. The techniques may further translate data values obtained from a source system into formats that meet the restrictions of a data structure used by a destination system.

Some embodiments use a data structure definition of a destination system to transmit data from a source system to structured data records of a destination system. A data transmission system uses a map associating source system fields with destination system fields in conjunction with the data structure definition. The data transmission system then reads values from the source system fields and stores them in the mapped destination system fields. The system may thus automatically port unstructured data from the source system to appropriate locations in the structured data records of the destination system. For example, the system may port abstracted data associated with subjects into structured data records of an EDC system for use in clinical studies.

Some embodiments use a data structure definition of a destination system to translate data from a source system into a target format for the destination system. A data transmission system identifies translation information for a particular field stored in the data structure definition and uses the translation information to translate a respective data value from the source system. For example, the translation information may specify an encoding from candidate data values of the source system into respective candidate values of the destination system. The data transmission system may use the encoding to encode a data value obtained from the source system into a format of the destination system. The system thus automatically formats data from the source system into an appropriate format for the destination system. For example, the system may format abstracted data from an abstraction system into a target format of an EDC system.

Some embodiments provide system for ingesting data from a plurality of source systems into a destination system (e.g., an abstraction system), the plurality of source systems storing the data in conformance with a common specification (e.g., the Fast Healthcare Interoperability Resources (FHIR) specification) indicating a standard for transmitting datasets, the plurality of source systems including a first source system (e.g., a first EHR system) and a second source system (e.g., a second EHR system). The system may be configured to request data from the plurality of source systems by: (1) generating a first query requesting a first dataset stored in the first source system, the first query including a first value of at least one field designated by the common specification for referencing a dataset; and (2) generating a second query requesting a second dataset stored in the second source system, the second query including a second value of the at least one field designated by the common specification for referencing a dataset. The system may be configured to communicate, through a communication network (e.g., the Internet), with the plurality of source systems. The system may be configured to: (1) transmit, through the communication network, the first query to the first source system and the second query to the second source system; and (2) receive, through the communication network, after transmission of the first query and the second query, the first dataset (e.g., a first set of CVN documents) from the first source system; and the second dataset (e.g., a second set of CVN documents) from the second source system. The system may be configured to store, in a datastore accessible by the destination system, the first dataset and the second dataset.

In some embodiments, the at least one field designated by the specification for uniquely referencing the dataset comprises a subject identifier that uniquely identifies a subject that the dataset is associated with in a particular source system.

In some embodiments, the system may comprise memory storing, for each of a plurality of subjects, respective values of the subject identifier used by the plurality of source systems to identify the subject. The system may be configured configured to determine the first value of the at least one field and the second value of the at least one field by: (1) identifying, in the memory, a value of the subject identifier used by the first source system to uniquely reference a first subject, wherein the first dataset is associated with the first subject in the first source system; and (2) identifying, in the memory, a value of the subject identifier used by the second source system to uniquely reference the first subject, wherein the second dataset is associated with the first subject in the second source system.

In some embodiments, the system may be configured to: (1) identify, from among a plurality of subjects, a subject with a destination system identifier for the subject that is associated with an identifier for the subject used by the first source system; and (2) trigger generation of the first query to request data from the first source system after identifying the subject.

In some embodiments, the system may comprise memory storing sets of authentication credentials for the plurality of source systems. The system may be configured to: (1) authenticate the system to the first source system using a first one of the sets of authentication credentials for the first source system to obtain a first access token; (2) authenticate the system to the second source system using a second one of the sets of authentication credentials for the second source system to obtain a second access token. The system may be configured to transmit, through the communication network, the first query to the first source system and the second query to the second source system by: (1) transmitting, through the communication network, the first query to the first source system using the first access token; and (2) transmitting, through the communication network, the second query to the second source system using the second access token.

In some embodiments, the first dataset is associated with a first subject in the first source system and the second dataset is associated with the first subject in the second source system. Determining the first value of the at least one field comprises determining a value of the at least one field used by the first source system to uniquely identify the first subject in the first source system. Determining the second value of the at least one field comprises determining a value of the at least one field used by the second source system to uniquely identify the first subject in the second source system.

In some embodiments, the first dataset is associated with a first one of the plurality of subjects and the second dataset is associated with a second one of the plurality of subjects. Determining the first value of the at least one field comprises determining a value of the at least one field used by the first source system to uniquely identify data associated with the first subject in the first source system. Determining the second value of the at least one field comprises determining a value of the at least one field used by the second source system to uniquely identify data associated with the second subject in the second source system.

In some embodiments, the system may be configured to store, in the datastore, the first dataset and the second dataset by performing: read the first dataset from the first source system into a cloud-based datastore, separate from the datastore of the system; read the second dataset from the second source system into the cloud-based datastore; and read the first dataset and the second dataset from the cloud-based datastore into the datastore of the destination system.

In some embodiments, the system may be configured to periodically request data associated with a plurality of subjects from the plurality of source systems.

Some embodiments provide a data transmission system for transmitting data from a source system (e.g., an abstraction system storing data abstracted from CVN documents associated with subjects) into data records of a destination system (e.g., an EDC system). The system may be configured to access, from the destination system, a data structure definition (e.g., a JSON template) for the data records of the destination system. The data structure definition may indicate: (1) a plurality of destination system fields for storing values in the data records; and (2) translation information for at least one of the plurality of destination system fields, the translation information comprising information for translating data obtained from the source system for storage in the at least one destination system field. The system may be configured to transmit, using the data structure definition, data from a source system data record (e.g., an SQLite file) to a destination system data record having a structure defined by the data structure definition by: (1) identifying, using a field map that associates source system fields with respective ones of the plurality of destination system fields, at least one source system field from which to read a value; (2) accessing a value of the at least one source system field from the source system data record; (3) translating, using the translation information for the at least one destination system field in the data structure definition, the value of the at least one source system field to obtain a translated value (e.g., by formatting the value stored in the at least one source system field into the target format of the at least one destination system field and/or converting the data into a structured representation for the at least one destination system field); and (4) storing the translated value in the at least one destination system field of the destination system data record.

In some embodiments, the data structure definition includes a container that includes the plurality of fields. In some embodiments: the field map associates the source system data record with the container of the data structure definition. The transmitting comprises: (1) identifying, in the field map, the container of the data structure definition associated with the source system data record; (2) accessing, from the data structure definition, the plurality of fields in the container; and (3) identifying, using the field map, the at least one source system field associated with the at least one destination system field after accessing the plurality of fields in the container.

In some embodiments, the translation information for the field comprises: a plurality of candidate values of the at least one source system field; a plurality of translated values corresponding to the plurality of candidate values. Translating, using the translation information for the at least one destination system field, the value of the at least one source system field to obtain the translated value comprises: (1) identifying one of the plurality of candidate values that is stored in the at least one source system field; and (2) selecting the corresponding one of the plurality of translated values as the translated value for the at least one destination system field.

In some embodiments, storing the translated value in the at least one destination system field of a respective one of the data records associated with the subject comprises: (1) generating an operational data model (ODM) extensive markup language (XML) file including the translated value; and (2) transmitting the ODM XML file to the destination system for storage.

In some embodiments, transmitting the data from the source system data record to the destination system data record comprises transmitting unstructured data from the source system data record to the destination system data record.

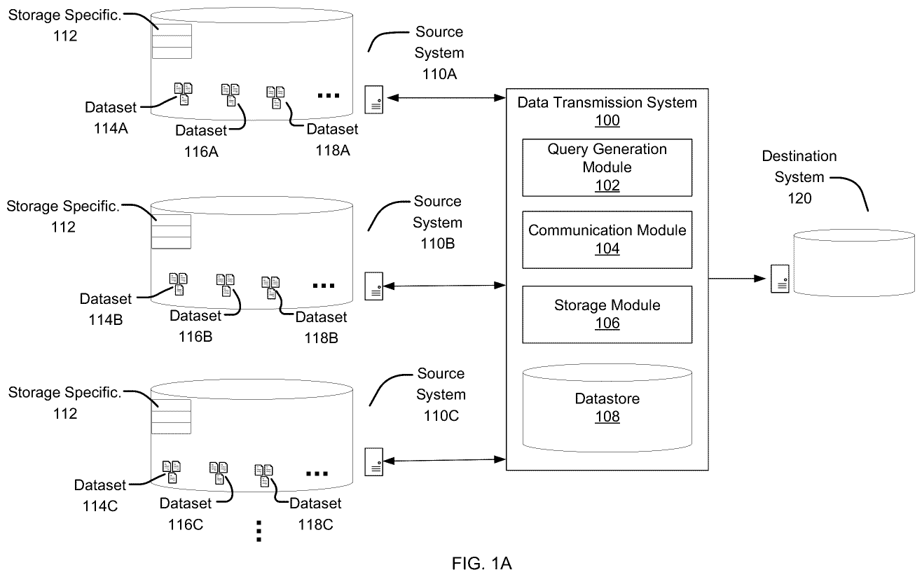

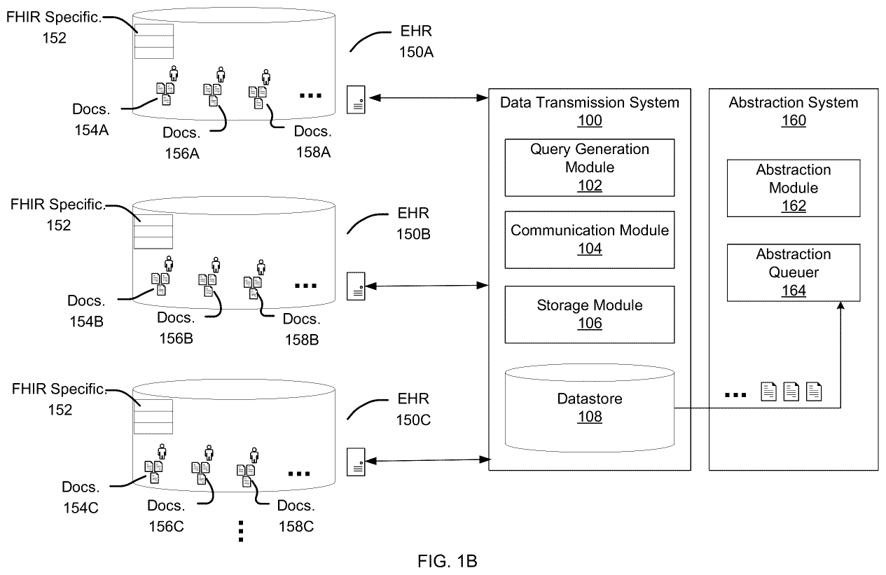

FIG. 1A is an example environment in which some embodiments of the technology described herein may be implemented. The environment includes a data transmission system 100 in communication with multiple source systems 110A, 110B, 110C and a destination system 120. The data transmission system 100 may transmit data from the source systems 110A, 110B, 110C to the destination system 120. In some embodiments, the data transmission system 100 may be implemented as part of the destination system 120 to ingest data into the destination system 120 from the source systems 110A, 110B, 110C.

As shown in FIG. 1A, each of the source systems 110A, 110B, 110C includes a datastore (e.g., a database) in which the source system stores respective sets of data. Source system 110A stores datasets 114A, 116A, 118A, source system 110B stores datasets 114B, 116B, 118B, and source system 110C stores datasets 114C, 116C, 118C. As illustrated in FIG. 1A, each of the datasets 114A, 114B, 114C, 116A, 116B, 116C, 118A, 118B, 118C may include one or more data records. In some embodiments, a dataset may include unstructured data. For example, a given dataset may include one or more documents (e.g., CVN documents storing physician notes). In some embodiments, a given dataset may store unstructured data. In some embodiments, a dataset may include a combination of structured and unstructured data.

In some embodiments, the data stored by each of the source systems 110A, 110B, 110C may be dynamic. For example, each of the source systems 110A, 110B, 110C may be an EHR system that is frequently being updated with clinical record data by clinicians (e.g., physicians, nurses, and/or other clinicians). As such, the data may regularly change over time.

As indicated in FIG. 1A, each of the source systems 110A, 110B, 110C stores its datasets in conformance with a common specification 112. The specification 112 may indicate rules for defining datasets (e.g., for transmission). In some embodiments, the rules may include rules for defining a dataset, referencing the dataset, storing the dataset, and/or metadata to be stored about the dataset. The specification 112 may specify a rule for uniquely identifying datasets in storage of each source system. In some embodiments, the specification 112 may specify a structure including one or more fields that store information about a dataset. For example, the field(s) may include a field configured to store a unique identifier for the dataset. As another example, the field(s) may include a field configured to store an identifier of a subject associated with the dataset. As another example, the field(s) may include a field configured to store a description of the dataset.

As shown in FIG. 1A, each of the source systems 110A, 110B, 110C includes a computing device (e.g., a server). The computing device may perform data processing operations on data in storage of the source system 110A, 110B, 110C. In some embodiments, the data processing operations may include executing queries (e.g., received from the data transmission system 100). In some embodiments, the server may communicate with the data transmission system 100. The server may transmit and receive communications from the data transmission system 100. For example, the server may exchange communications with the data transmission system 100 to authenticate the data transmission system 100, receive queries, and/or transmit responses to queries. In some embodiments, the computing device may communicate with the data transmission system 100 through a communication network. For example, the communication network may be a wireless communication network (e.g., the Internet).

As shown in FIG. 1A, the data transmission system 100 includes multiple modules that the data transmission system 100 uses to access data from the source systems 110A, 110B, 110C. The data transmission system 100 includes a query generation module 102, a communication module 104, a storage module 106, and a datastore 108.

In some embodiments, the query generation module 102 may generate queries for requesting data from the source systems 110A, 110B, 110C. The query generation module 102 may generate the queries based on the specification 112 according to which the source systems 110A, 110B, 110C store data. In some embodiments, the query generation module 102 may generate a query requesting data from a particular source system by: (1) determining a value of one or more fields designated by the specification 112 to uniquely identify a dataset; (2) include the value of the field(s) in a query requesting the data. For example, the query generation module 102 may determine a value of a subject identifier field designated by the specification 112 to reference a collection of documents (e.g., CVN documents) associated with the subject. The query generation module 102 may generate a query requesting the collection of documents associated with the subject that includes a value of the subject identifier field associated with the subject. The query, when executed by one of the source systems 110A, 110B, 110C (e.g., by a computing device of the source system), may cause the source system to return a dataset including the requested collection of documents and/or a reference to the requested collection of documents.

In some embodiments, the query generation module 102 may generate queries for the different source systems 110A, 110B, 110C using the same process. The query generation module 102 may generate the queries based on the specification 112 that is common across the different source systems 110A, 10B, 110C. In some embodiments, the query generation module 102 may generate the queries by including, in each query, a value of field(s) designated by the specification 112 to uniquely identify data. For example, to obtain CVN documents associated with a particular subject stored in source systems 110A, 110B, the query generation module 102 may generate: (1) a first query including a request for CVN documents that includes a value of a subject identifier field used by the source system 110A to identify the particular subject; and (2) generate a second query including a request for CVN documents that includes a value of the subject identifier field used by the source system 110B to reference a particular subject. Accordingly, the query generation module 102 may generate queries in a uniform manner across all the source systems 110A, 110B, 110C.

The communication module 104 may communicate with the source systems 110A, 110B, 110C. The communication module 104 may communicate with the source systems 110A, 110B, 110C through a communication network (e.g., the Internet). In some embodiments, the communication module 104 may establish a communication channel with a given source system. The communication module 104 may establish the communication channel by authenticating the data transmission system 100 to the source system. The communication module 104 may authenticate the data transmission system 100 to a source system by providing credentials (e.g., a username and password obtained from previously registering the data transmission system 100 with the source system) to the source system. For example, the communication module 104 may receive an access token in response to providing valid credentials to the source system. The communication module 104 may use the access token to communicate with the source system.

In some embodiments, the communication module 104 may communicate with the source systems 110A, 110B, 110C using respective credentials for each source system. For example, the communication module 104 may access (e.g., from datastore 108) credentials (e.g., an id and/or secret) for the source systems 110A, 110B, 110C and communicate with each of the source systems 110A, 110B, 110C using the credentials designated for the source system. In some embodiments, the communication module 104 may communicate with each of the source systems 110A, 110B, 110C through a communication network by accessing network locations of the source systems 110A, 110B, 110C. For example, the communication module 104 may access a URL or address associated with a source system (e.g., from the datastore 108), and transmit communications to the URL or address. To illustrate, the communication module 104 may communicate with an authentication server of a source system using a first URL associated with the authentication server and communicate with a data server of the source system using a second URL associated with the data server.

In some embodiments, the communication module 104 may authenticate the data transmission system 100 to a source system using an authentication protocol. For example, the communication module 104 may authenticate the data transmission system 100 to a source system using the OAuth 2 protocol, a Kerberos protocol, or other suitable authentication protocol. In some embodiments, the communication module 104 may authenticate the data transmission system 100 to different source systems using different respective authentication protocols. For example, the communication module 104 may authenticate the data transmission system 100 to source system 110A with a first authentication protocol and a first set of credentials, and to source system 110B with a second authentication protocol and a second set of credentials.

In some embodiments, the communication module 104 may periodically communicate with the source systems 110A, 110B, 110C to obtain data (e.g., to obtain updated data). For example, the communication module 104 may periodically transmit queries requesting new sets of CVN documents associated with subjects. The communication module 104 may periodically transmit a query to a given source system every day, every week, every month, or other suitable frequency.

The storage module 106 may store data obtained from the source systems 110A, 110B, 110C in response to queries transmitted to the source systems 110A, 110B, 110C. In some embodiments, the storage module 106 may organize ingested data by subject. For example, the storage module 106 may store, for each of multiple subjects, CVN documents obtained for the subject in response to queries. In some embodiments, the storage module 106 may store data obtained from the source systems 110A, 110B, 110C in cloud-based storage (e.g., an Amazon S3 cloud object).

In some embodiments, the storage module 106 may retrieve data from the datastore 108 and output the data to another module or system. For example, the storage module 106 may output data from the datastore 108 into a queue. To illustrate, the storage module 106 may output CVN documents stored in the datastore 108 into a queue of an abstraction system through which abstractors may analyze the CVN documents to extract data points for subjects.

The datastore 108 of the data transmission system 100 may comprise storage hardware (e.g., hard drive(s)) configured to store data ingested from the source systems 110A, 110B, 110C. In some embodiments, the datastore 108 may be a cloud-based data storage system. Although illustrated as being within the data transmission system 100 in FIG. 1A, in some embodiments, the cloud-based data storage system may comprise multiple distributed data storage components. In some embodiments, the datastore 108 may comprise a database controlled by a database management system (DBMS).

The destination system 120 may be a system that the data transmission system 100 provides data to. The destination system 120 may receive data ingested by the data transmission system 100. For example, the destination system may receive CVN documents associated with subjects that were obtained by the data transmission system 100 (e.g., by transmitting queries to the source systems 110A, 110B, 110C). As shown in FIG. 1A, the destination system 120 includes a datastore (e.g., a database) and a computing device (e.g., a server) for executing computations using data in the datastore.

FIG. 1B illustrates an example implementation of the data transmission system 100 for use in ingesting data from EHR systems 150A, 150B, 150C into an abstraction system 160, according to some embodiments of the technology described herein. In the example of FIG. 1B, the data transmission system 100 obtains documents associated with various subjects from the EHR systems 150A, 150B, 150C for use by the abstraction system 160. The abstraction system 160 may be used to extract information about subjects (e.g., patients) from documents (e.g., CVN documents) associated with the subjects. The documents may store unstructured data (e.g., physician notes) that needs to be further analyzed in order to extract data points about the subjects. Example data points that may be extracted about a subject from documents may include menopausal status, pregnancy status, prior therapy (e.g., prior cancer therapy), smoking status, and/or other data points.

In the environment of FIG. 1B, each of the EHR systems 150A, 150B, 150C stores documents associated with respective subjects. For example, EHR 150A stores documents 154A associated with a first subject, documents 156A associated with a second subject, and documents 158A associated with a third subject. EHR 150B stores documents 154B associated with the first subject, documents 156B associated with the second subject, and documents 158B associated with the third subject. EHR 150C stores documents 154C associated with the first subject, documents 156C associated with the second subject, and documents 158C associated with the third subject.

In the example of FIG. 1B, each of the EHR systems 150A, 150B, 150C stores the documents in accordance with the FHIR specification. According to the FHIR specification, for each set of documents associated with a subject, an EHR system may store a reference (e.g., a “Bundle”) to the set of documents. The documents may be stored in units (e.g., “DocumentReference” units). To illustrate, a “Bundle” resource in FHIR may contain “DocumentReference” resource(s) that each includes content from one or more documents stored in the EHR system.

In some embodiments, the query generation module 102 may generate queries requesting documents associated with respective subjects. The query generation module 102 may generate a query for an EHR system requesting a reference to documents associated with a subject by specifying a subject identifier (e.g., a value of the “patient” field). In response to the query, the EHR system may provide the reference to one or more datasets storing the documents. For example, the EHR system may provide a “Bundle” resource containing one or more “DocumentReference” resources that each includes content of one or more documents. The data transmission system 100 may store content from the “DocumentReference” resource(s) in the datastore 108. As another example, the storage module 106 may save link(s) to the “Document Reference” resource(s) in the datastore 108 (e.g., in a cloud-based datastore). The link(s) may then be accessed to obtain documents from the EHR system.

In some embodiments, the datastore 108 may store documents obtained from the EHR systems 150A, 150B, 150C in the datastore 108 in association with respective subjects. For example, the storage module 106 may store documents associated with a first subject and obtained from the EHR system 150A in association with an identifier of the first subject in the datastore 108. The storage module 106 may store documents associated with the first subject and obtained from the EHR system 150B in association with the same identifier in the datastore 108. Thus, the data transmission system 100 may aggregate documents associated with a given subject from multiple different EHR systems in the datastore 108.

As illustrated in FIG. 1B, the abstraction system 160 includes an abstraction module 162 and an abstraction queuer 164.

In some embodiments, the abstraction module 162 may extract data points from documents. For example, the abstraction module 162 may generate a GUI displaying information from documents associated with a subject and providing an interactive interface through which a human abstractor can input extracted data points. As another example, the abstraction module 162 may use machine learning to process documents to automatically extract data points about subjects.

In some embodiments, the data transmission system 100 may transfer datasets (e.g., including documents) from the datastore 108 to a datastore of the abstraction system 160. The data transmission system 100 may request that the abstraction queuer 164 queue a subject for abstraction. When new dataset(s) have been ingested for a subject, the abstraction queuer may generate, in response to the request, an abstraction task for the abstraction module 162 to extract specific data points from the new dataset(s). Accordingly, the data transmission system 100 may trigger abstraction by the abstraction module 162. In some embodiments, the data transmission system 100 provides an automated pipeline that obtains documents from EHR systems 150A, 150B, 150C for use by the abstraction system 160. In some embodiments, the abstraction system 160 may include a datastore (e.g., memory) for storing datasets received from the data transmission system 100 and for storing abstracted data.

In some embodiments, the data transmission system 100 may cause the abstraction queuer 164 to automatically initiate abstraction by storing document(s) in a datastore of the abstraction system 160. For example, the data transmission system 100 may trigger generation of a GUI through which an abstractor can input extracted points. As another example, the data transmission system 100 may trigger an automated extraction process performed by the abstraction system 160 (e.g., using one or more machine learning models to process documents).

In some embodiments, the abstraction system 160 may store an identifier uniquely identifying a subject. For example, the identifier may be a subject ID associated with the subject from an electronic data capture (EDC) system to which data from the abstraction system 160 is to be transmitted. The datastore 108 may store a mapping between the subject's identifier(s) in the EHR systems 150A, 150B, 150C and the abstraction system's 160 identifier for the subject. The abstraction system 160 may store data associated with a subject using the abstraction system identifier for the subject. The data transmission system 100 may store data associated with a given subject in a datastore of the abstraction system 160 in association with the abstraction system's identifier for the subject. In some embodiments, data transmission system 100 may use the mapping to determine identifier(s) for the subject in the EHR systems 150A, 150B, 150C, and use the determined identifier(s) to query the EHR systems 150A, 150B, 150C for documents associated with the subject.

Although in FIG. 1B the data transmission system 100 is shown separately from the abstraction system 160, in some embodiments, the data transmission system 100 may be implemented as part of the abstraction system 160. For example, the data transmission system 100 may be implemented as a data ingestion module of the abstraction system 160. In some embodiments, the data transmission system 100 may be separate from the abstraction system 160 as illustrated in FIG. 1B. The abstraction system 160 may communicate with the data transmission system 100 (e.g., through a communication network).

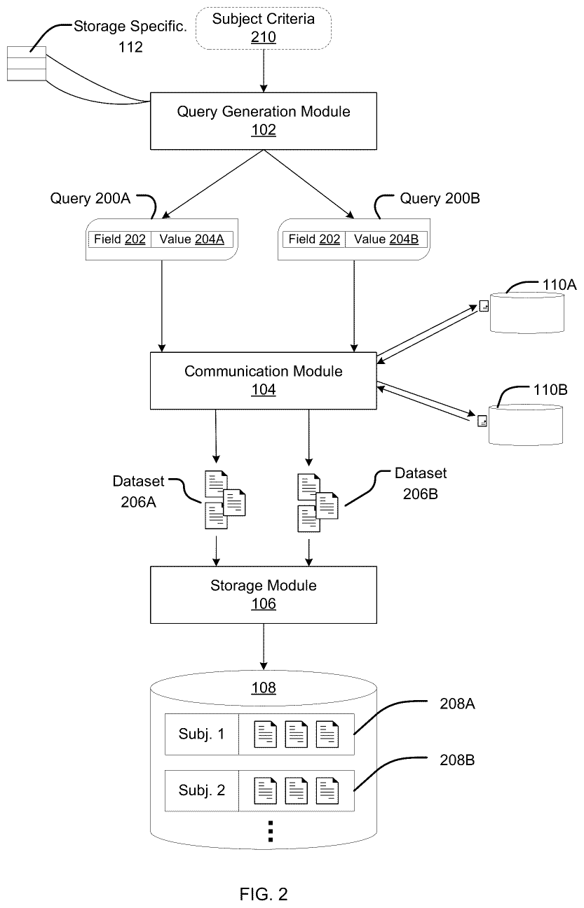

FIG. 2 illustrates operation of the data transmission system 100 for obtaining datasets from source systems 110A, 110B, according to some embodiments of the technology described herein.

As shown in FIG. 2, in some embodiments, the query generation module 102 may obtain a set of subject criteria 210. The query generation module 102 may use the subject criteria 210 to identify, from among multiple subjects for whom the source systems 110A, 110B, 110C store data, a set of subjects for whom to obtain datasets (e.g., documents). For example, the subject criteria 210 may indicate a criterion that requires a subject to have opted into a clinical study. As another example, the subject criteria 210 may indicate a criterion that requires a subject to have a mapping from an EHR system identifier of the subject to an EDC system identifier for the subject. The datastore 108 of the data transmission system 100 may store information about subjects that can be used to determine whether a subject meets the criteria 210. For example, the datastore 108 may store an opt-in status for subjects which may be used by the query generation module 102 to determine which subjects are opted into a clinical study. As indicated by the dashed line around the criterion 210, the query generation module 102 is not required to receive or use subject criteria 210.

As shown in FIG. 2, the query generation module 102 generates query 200A for the first source system 100A and query 200B for the second source system 110B. For example, the query 200A may be for requesting a dataset associated with a first subject from the source system 110A and the query 200B may be for requesting a dataset associated with the first subject from the source system 110B. As another example, the query 200A may be for requesting a dataset associated with a first subject from the source system 110A and the query 200B may be for requesting a dataset associated with a second subject from the source system 110B. Each of the queries 200A, 200B indicates a value of a field 202. For example, the field 202 may be a subject identifier field. The value 204A may be a value of the field 202 for the first source system 110A and the value 204B may be a value of the field 202 for the second source system 110B.

The communication module 104 transmits the query 200A to the source system 110A (e.g., through a communication network using a first URL associated with the source system 110A) and the query 200B to the source system 110B (e.g., through a communication network using a second URL associated with the source system 110B). The queries 200A, 200B may be executed by respective source systems 110A, 110B. The source systems 110A, 110B may transmit (e.g., through the communication network) respective datasets 206A, 206B to the communication module 104 in response to executing the queries 200A, 200B. For example, the dataset 206A may be documents associated with a first subject that are returned by the source system 110A and the dataset 206B may be documents associated with the first subject that are returned by the source system 110A. As another example, dataset 206A may be documents associated with a first subject that are returned by the source system 110A and the dataset 206B may be documents associated with a second subject that are returned by the source system 110B.

As shown in FIG. 2, the storage module 106 stores the datasets 206A, 206B in the datastore 108. In the example of FIG. 2, the storage module 106 stores each dataset in association with a respective subject. The entry 208A in the datastore 108 includes the dataset 206A stored in association with an identifier of a first subject. The entry 206B in the datastore 108 includes the dataset 206B stored in association with an identifier of a second subject.

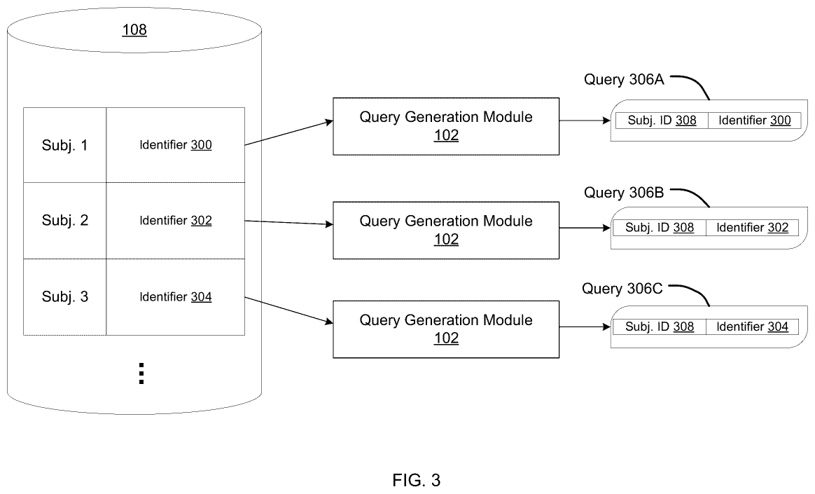

FIG. 3 shows an example of how the query generation module 102 generates queries using subject identifiers stored in the datastore 108, according to some embodiments of the technology described herein. As shown in FIG. 1, the datastore 108 stores, for each of the subjects, a respective set of identifiers. The identifiers include: identifier 300 a first subject, identifier 302 for a second subject, and identifier 304 for a third subject. Each subject identifier may uniquely identify the subject in a particular source system. For example, identifier 300 may be an identifier of the first subject in the source system 110A and identifier 302 may be an identifier of the second subject in the source system 110B.

In the example of FIG. 3, the query generation module 102 generates queries 306A, 306B, 306C. Each of the queries 306A, 306B, 306C requests data associated with a particular subject. Accordingly, each of the queries 306A, 306B, 306C specifies a value of a subject identifier field 308 (e.g., specified by a specification). The query generation module 102 may generate the query 306A to request data associated with the first subject from the source system 110A. Accordingly, the query 306A includes the identifier 300 as the value of the subject identifier field 308. The query generation module 102 may generate the query 306B to request data associated with the second subject from the source system 110B. Accordingly, the query 306B includes the identifier 302 as the value of the subject identifier field 308. The query generation module 102 may generate the query 306C to request data associated with the third subject from the source system 110C. Accordingly, the query 306C includes the identifier 304 as the value of the subject identifier field 308.

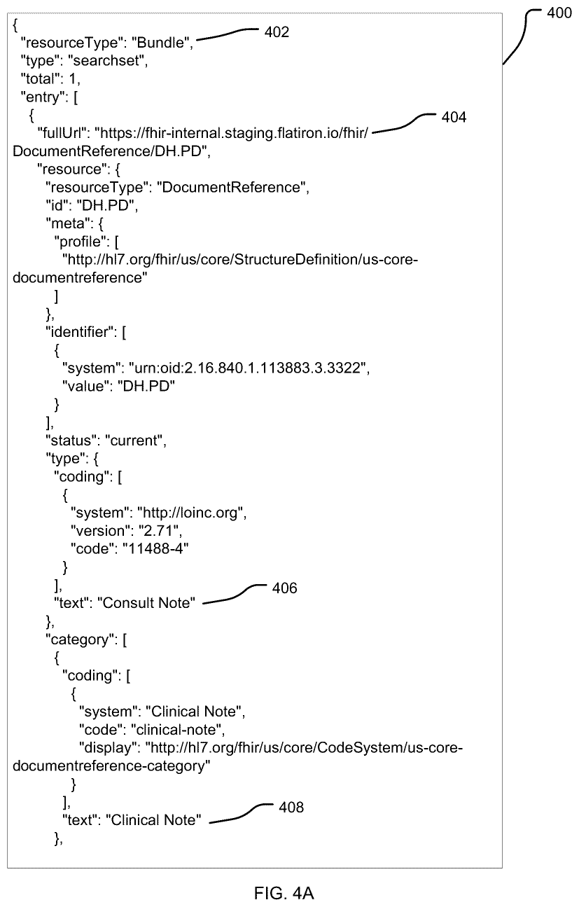

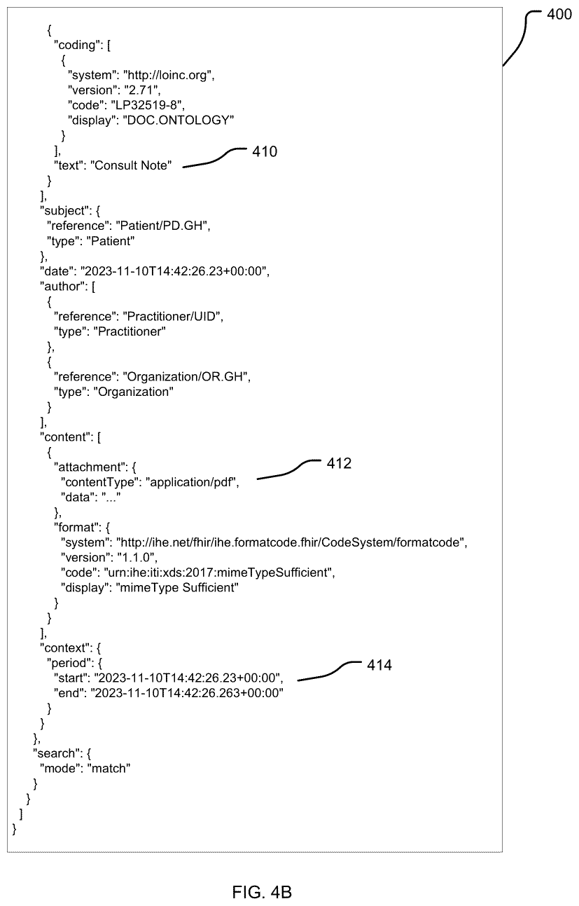

FIGS. 4A-4B show an example response 400 to a query generated by the data transmission system 100, according to some embodiments of the technology described herein. In the example of FIG. 4, the response 400 is a JSON file that is obtained using a query. The JSON file includes a Bundle resource which contains multiple “DocumentReference” resources. For example, the response 400 may be to the following query: “https://flatiron.com/fhir/DocumentReference?patient=fakePatientIdentifier123”. As shown in FIG. 4, the response 400 is a resource bundle as indicated by the “resourceType” attribute 402 in the response 400. The response 400 includes a URL 404 for documents. The response 400 includes “DocumentResource” resources including content from CVN documents 406, 408, 410. The response 400 further specifies a PDF attachment 412. The response 400 includes an indication 414 of a time at which the query was executed.

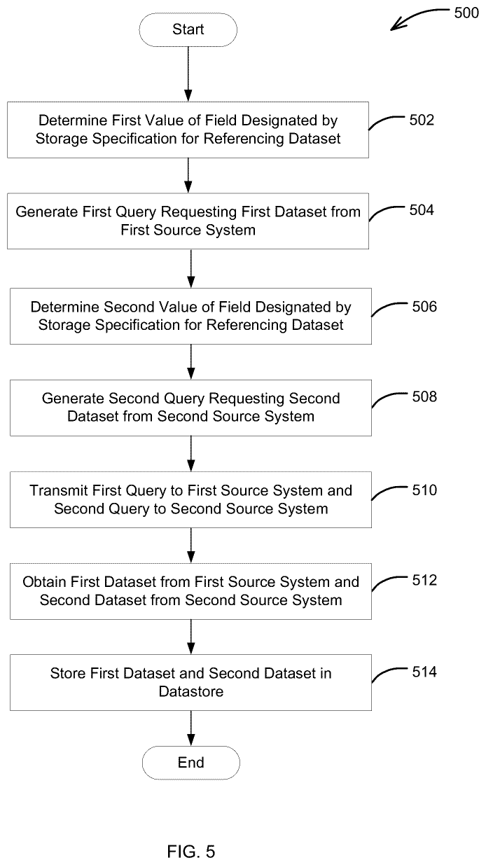

FIG. 5 is an example process 500 for ingesting data into a destination system from multiple different source systems, according to some embodiments of the technology described herein. In some embodiments, process 500 may be performed by the data processing system 100 described herein. The source systems may store data in accordance with a common specification. For example, process 500 may be performed by the data processing system 100 to ingest unstructured data (e.g., documents) from multiple EHR systems that store data in accordance with the FHIR specification.

Process 500 begins at block 502, where the system determines a first value of a field designated by the specification for referencing a dataset. In some embodiments, the field may be a field within a particular resource defined by the specification. The field may reference a dataset associated with a subject. As an illustrative example, the field may be the patient field of the DocumentReference resource of the FHIR standard. In this example, the patient field may be used to identify a set of one or more DocumentReference resources associated with a subject. The system may determine the first value of the field for a first source system (e.g., a first EHR system). In some embodiments, the system may store, in a datastore, values of the field used by different source systems to identify a subject. The system may determine the first value of the field for the first source system by identifying, in the datastore, a value of the field used by the first source system to uniquely identify a subject. For example, the datastore may store patient field values for the subject used by each of the source systems. The system may determine the first value of the field to be the patient field value identifying a subject used by the first source system.

Next, process 500 proceeds to block 504, where the system generates a first query requesting a first dataset from the first source system. For example, the first dataset may be document(s) associated with a particular subject stored in the first source system. In some embodiments, the system may generate the first query by: (1) generating a query statement; and (2) including the first value of the field in the query statement to request the first dataset. For example, the system may generate an HTTP query specifying a first value of the patient field to request documents associated with a first subject stored in the first source system.

Next, process 500 proceeds to block 506, where the system determines a second value of the field (e.g., for the same or a different subject). The system may determine the second value of the field for a second source system (e.g., a second EHR system). In some embodiments, the system may determine the second value of the field for the second source system by identifying, in a datastore of the system, a value of the field associated with the second source system. For example, the system may determine the second value of the field to be the patient field value identifying a subject used by the second source system.

Next, process 500 proceeds to block 508, where the system generates a second query requesting a second dataset from the second source system. For example, the second dataset may be document(s) associated with a subject stored in the second source system. In some embodiments, the system may generate the second query by: (1) generating a query statement; and (2) including the second value of the field in the query statement to request the second dataset. For example, the system may generate an HTTP query specifying a second value of the patient field to request documents associated with the first subject or a second subject stored in the second source system.

Next, process 500 proceeds to block 510, where the system transmits the first query to the first source system and the second query to the second query system. In some embodiments, the system may transmit the queries through a communication network (e.g., the Internet). In some embodiments, the system may transmit a query to a respective source system after the system is authenticated to the respective source system (e.g., using credentials for the respective source system stored by the system). For example, the system may obtain an access token from the respective source system and transmit the query to the respective source system using the access token.

Next, process 500 proceeds to block 512, where the system obtains the first dataset from the first source system and the second dataset from the second source system. The transmitted first query may have been executed by the first source system against its stored data and the transmitted second query may have been executed by the second source system against its stored data. The system may obtain the results obtained from execution of the first and second queries against respective source systems. For example, the system may receive a first collection of documents associated with a subject and stored in the first source system, and a second collection of documents associated with the subject stored in the second source system. As another example, the system may receive a first collection of documents associated with a first subject and stored in the first source system, and a second collection of documents associated with a second subject and stored in the second source system.

In some embodiments, the system may obtain the first dataset and the second dataset from respective source systems as responses to the first and second queries by the first and second source systems. For example, the system may obtain the first dataset from the first source system after execution of the first query by the first source system (e.g., in a “Bundle” resource specified by the FHIR standard). As another example, the system may obtain the second dataset from the second source system after execution of the second query by the second source system (e.g., in a “Bundle” resource specified by the FHIR standard).

Next, process 500 proceeds to block 514, where the system stores the first dataset and the second dataset in a datastore accessible by the destination system. In some embodiments, the system may store the first dataset and the second dataset in a datastore of the destination system. For example, the system may store the first dataset and the second dataset in a datastore of an abstraction system (e.g., abstraction system 160). In some embodiments, the system may store the first dataset and the second dataset in a datastore external to the destination system that can be accessed by the destination system. For example, the first and second datasets may include documents associated with a subject. The system may store the documents in association with an identifier of the subject in the datastore. As another example, the first dataset may include documents associated with a first subject and the second dataset may include documents associated with a second subject. The system may store documents associated with the first subject in the datastore in association with an identifier of the first subject (e.g., in a field designated for the first subject in the datastore). The system may store documents associated with the second subject in the datastore in association with an identifier of the second subject (e.g., in a field designated for the second subject in the datastore).

In some embodiments, the system may trigger one or more functions in a destination system (e.g., abstraction system 160 described herein with reference to FIG. 1B). The system may trigger the function(s) by storing the first dataset and/or the second dataset in a datastore of the destination system. For example, the system may trigger abstraction of data points for subject(s) from the first and second datasets. In some embodiments, the system may trigger the presentation of the first and second datasets in a GUI provided by the abstraction system 160. The system may present the GUI in a display of a user device accessing the destination system. An abstractor may view a visualization of the data from the first and second datasets to perform abstraction using the GUI. In some embodiments, the system may trigger execution of automatic abstraction in which the destination system automatically processes the first and second dataset to extract data points from the datasets (e.g., by extracting values of clinical variables). For example, the system may trigger execution of automatic extraction that processes data from the first and/or second dataset using one or more machine learning models to abstract data. Example abstraction processes that may be triggered by the system (e.g., by storing the first and/or second dataset in a datastore) are described in U.S. patent application Ser. No. 17/963,998 filed on Oct. 11, 2022, which is incorporated by reference herein in its entirety.

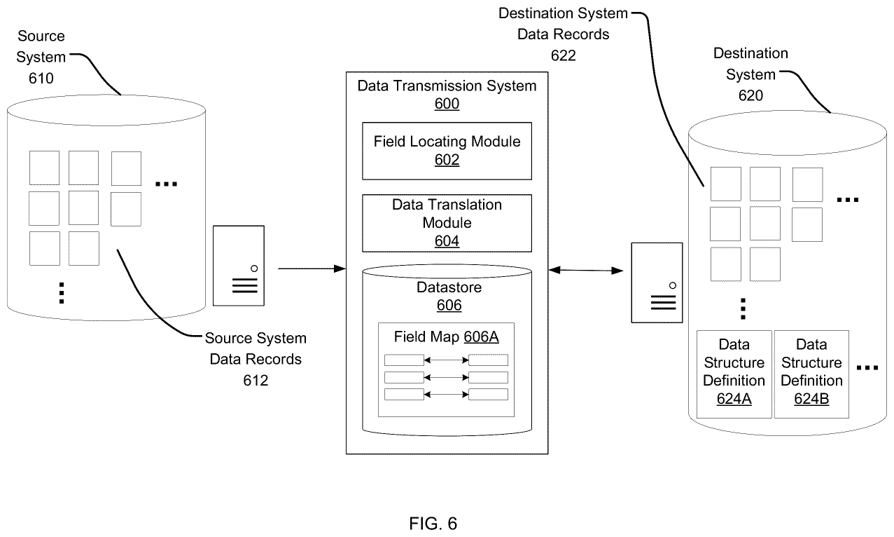

FIG. 6 shows an example environment in which a data transmission system 600 may operate, according to some embodiments of the technology described herein. As shown in FIG. 6, the environment includes a source system 610, the data transmission system 600, and a destination system 620.

As shown in FIG. 6, the source system 610 includes a datastore. The datastore may comprise storage hardware (e.g., hard drives) that is located at a particular location (e.g., a locally stored database) or distributed across multiple locations (e.g., a distributed database). The datastore may store information for the source system 610. In some embodiments, the source system 610 stores multiple data records 612. Each of the data records 612 may include one or more fields storing respective values. As an illustrative example, the source system 610 may be an abstraction system storing data values abstracted from processing documents (e.g., CVN documents) storing notes about subjects. The data values may be stored in fields of the data records 612. For example, the abstracted data may include answers to questions about a subject, or values of clinical variables (e.g., cancer type, menopausal status, medical condition diagnosis date, and/or other clinical variables). In some embodiments, each of the data records 612 may correspond to a form that includes one or more fields. The abstracted data for a given subject may be stored in fields of a form.

The source system 610 includes a computing device (e.g., a server) that performs processing using data stored in the datastore of the source system 610. The computing device may perform operations to generate data stored in the data records 612 and/or perform operations using the data stored in the data records 612. In some embodiments, the computing device may execute data access operations (e.g., read, update, delete) against the data records 612.

In some embodiments, the source system 610 may be the abstraction system 160 described herein with reference to FIG. 1B. For example, the abstraction system 160 may have processed documents obtained from an EHR to obtain data values that are stored in the data records 612.

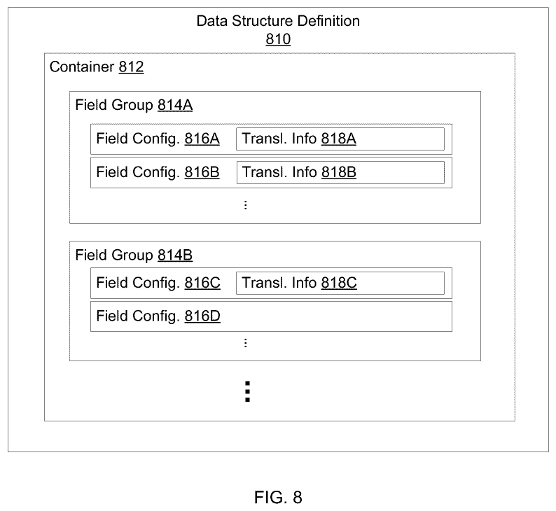

The environment of FIG. 6 includes a destination system 620 to which data from the source system 610 is transmitted by the data transmission system 600. The destination system 620 includes data records 622 for storing values. The data records 622 of the destination system 620 may be different from the data records 610 of the source system 610. In some embodiments, the data records 622 of the destination system 620 may each adhere to a particular data structure. As shown in FIG. 6, the destination system 620 may include data structure definitions 624A, 624B that each define a data structure. In some embodiments, a data structure definition may be a template according to which data is to be stored in records that adhere to the data structure. For example, the data structure definitions 624A 624B may each be a JSON template storing key, value pairs that describe the structure and fields of the data records. An example data structure definition is described herein with reference to FIG. 8.

In some embodiments, the destination system 620 may be an EDC system. The EDC system may need to obtain data values from the source system (e.g., an abstraction system) to store in its data records 622. The data values may form part of a set of data to be used in clinical studies (e.g., for pharmaceutical research, clinical trials, and/or other applications). The EDC system may require data to be stored according to a data structure defined by the data structure definition 624.

The data transmission system 600 includes a field locating module 602, a data translation module 604, and a datastore 606.

In some embodiments, the field locating module 602 may identify fields in the data records 622 of the destination system 620 at which to store data values obtained from the data records 612 of the source system. For a given source system field, the system may identify a corresponding destination system field to which to transmit a value of the source system field. For example, for a field in a form of the source system 610, the system may identify a corresponding field in a data structure used by the destination system 620.

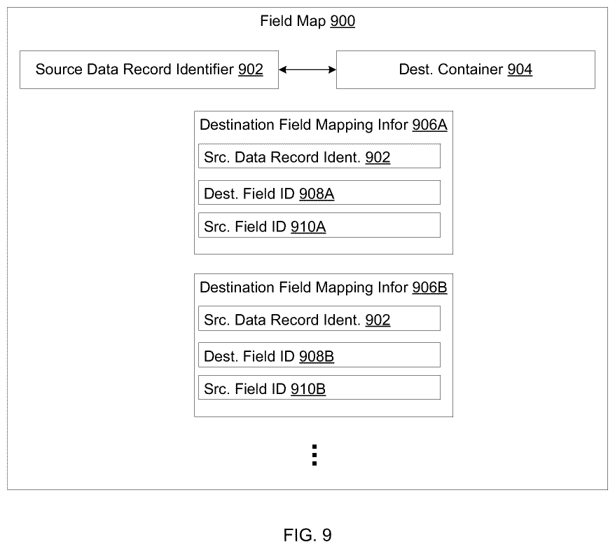

In some embodiments, the field locating module 602 may use a field map 606A to locate a destination system field in which to store a value of a source system field. The field map 606A may associate fields included in the source system data records 612 with fields in the destination system data records 622. To transmit data from a source system data record, the field location module 602 may use the field map 606A to: (1) identify a set of destination system field(s) to be populated from the source system data record; and (2) identify a mapping between the set of destination system field(s) and field(s) of the source system data record. An example field map is described herein with reference to FIG. 9.

In some embodiments, the data translation module 604 may translate a value of a source system field for storage in a destination system field. The data translation module 604 may access a data structure definition (e.g., one of data structure definitions 624A, 624B) of the destination system 620 and obtain translation information for the destination system field from the data structure definition. For example, the data structure definition may include, for a particular destination system field, translation information for translating a data value from a respective source system field into a valid format for the destination system field. In some embodiments, the translation information may be an encoding from a set of candidate source system field values to a corresponding set of destination system field values. For example, the translation information may include a mapping of candidate source system field values to respective candidate destination system field values. As another example, the translation information may specify a change in units. As another example, the translation information may be an encoding of source system field values into characters or codes. As another example, the translation information may encode a source system field value into a different range for the destination system field value. As another example, the translation information may change a case of characters in the source system field values.

The datastore 606 of the data transmission system 600 may include storage hardware (e.g., hard drive(s)). The datastore 606 may include memory of the data transmission system 600. As shown in FIG. 6, the datastore 606 stores the field map 606A.

In some embodiments, the destination system 620 may use data stored in the destination system 620 by the data transmission system 600 to perform further data processing. The destination system 620 may perform the data processing by accessing the data using one or more queries. A query may access the data by utilizing the data structure according to which it is stored in the destination system 620 (as a result of processing performed by the data transmission system 600). For example, the query may access abstracted data from fields specified in the data structure (e.g., by including filtering operations in the query that use the fields). Accordingly, the data transmission system 600 may allow the destination system 620 to more efficiently access data obtained from the source system 610 (e.g., because it is stored in a structured format of the destination system 620).

Although data transmission system 100 and 600 are described separately, in some embodiments, the data transmission system 100 and data transmission system 600 may be a single data transmission system. The modules of each of the systems 100, 600 may be integrated into the single data transmission system. Accordingly, the integrated data transmission system may be used to transmit data from source system(s) to destination system(s).

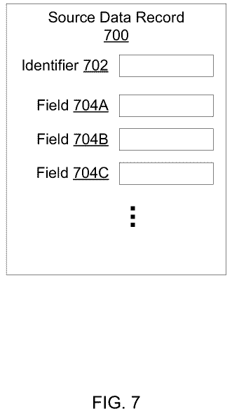

FIG. 7 is a diagram of an example source data record 700, according to some embodiments of the technology described herein. As shown in FIG. 7, the source data record 700 includes an identifier 702 and fields 704A, 704B, 704C. The identifier 702 may also be referred to as a “task definition”. In some embodiments, the source data record 700 may represent a form in a source system. For example, the source data record 700 may represent an abstraction form storing abstracted data. The identifier 702 may be an identifier of the form, and the fields 704A, 704B, 704C may be fields storing respective sets of information abstracted from processing documents associated with a subject.

In some embodiments, one or more of the fields 704A, 704B, 704C of the source data record 700 may be configured to store data that does not meet a structure of a corresponding field in the destination field. For example, a field value may not be formatted correctly, may not be expressed in the correct units, or may not be encoded in a code recognized in the destination system. In some embodiments, data in one or more of the fields 704A, 704B, 704C may be extracted from unstructured data (e.g., CVN documents).

FIG. 8 is a diagram of an example data structure definition 800, according to some embodiments of the technology described herein. As shown in FIG. 8, the data structure definition 800 includes a container 812 that holds field groups 814A, 814B. The container 812 may also be referred to as a “form” (e.g., because it represents a data record associated with a form in a destination system). Each of the field groups 814A, 814B may include one or more field configurations. A field configuration may indicate information about data stored in a respective destination system field. In the example of FIG. 8, the field group 814A includes field configurations 816A, 816B and field group 814B includes field configurations 816C, 816D. A field group may also be referred to as an “item group” and a field configuration may be referred to as an “item”.

In some embodiments, a field configuration for a destination system field may specify information about data to be stored in the destination system field. The field configuration may include translation information for translating data from a corresponding source system field for storage in the destination system field. In the example of FIG. 8, field configuration 816A includes translation information 818A, field configuration 818B includes translation information 818B and field configuration 816C includes translation information 818C. Examples of translation information are described herein. Field configuration 816D does not include translation information because its value may not need modification when transmitted to the destination system. Accordingly, a value of the destination field may be transmitted without translation from a source system field into the destination field.

In some embodiments, a field configuration may include information in addition or instead of translation information. For example, a field configuration may include a valid range of values for a destination system field, a data type (e.g., string, integer, float), an identifier for the field, a question being answered by the field value, and/or other information.

FIG. 9 is an example field map 900, according to some embodiments of the technology described herein. As shown in FIG. 9, the field map 900 includes a mapping between a source data record identifier 902 and a destination container 904 of a destination system's data structure definition. For example, the field map 900 may store a source data record identifier 902 in association with the name of a container in a data structure definition of a destination system. This may indicate that fields of a destination field data record that stored data according to the data structure definition are to be populated from the data record indicated by the source data record identifier 902.

The field map 900 includes mapping information for multiple destination system fields. In the example of FIG. 9, the field map 900 includes destination field mapping information 906A, 906B. Each set of destination field mapping information 906A, 906B may be associated with a particular destination field. As shown in FIG. 9, the destination field mapping information 906A includes the source data record identifier 902, a destination field ID 908A, and a source field ID 910A (e.g., a field name in the source data record) of a source field mapped to the destination field. The destination field mapping information 906B includes the source data record identifier 902, a destination field ID 908B, and a source field ID 910B (e.g., a field name in the source data record) of a source field mapped to the destination field.

In some embodiments, destination field mapping information for a destination system field may include information in addition to an indication of a source field and a source data record identifier. For example, the destination field mapping information for the destination system field may include an attribute indicating that the field value is populated from a source system field. As another example, the destination field mapping information may include information about the destination system field obtained from a field configuration of the destination system field in a data structure definition. Example such information is described herein with reference to FIG. 8.

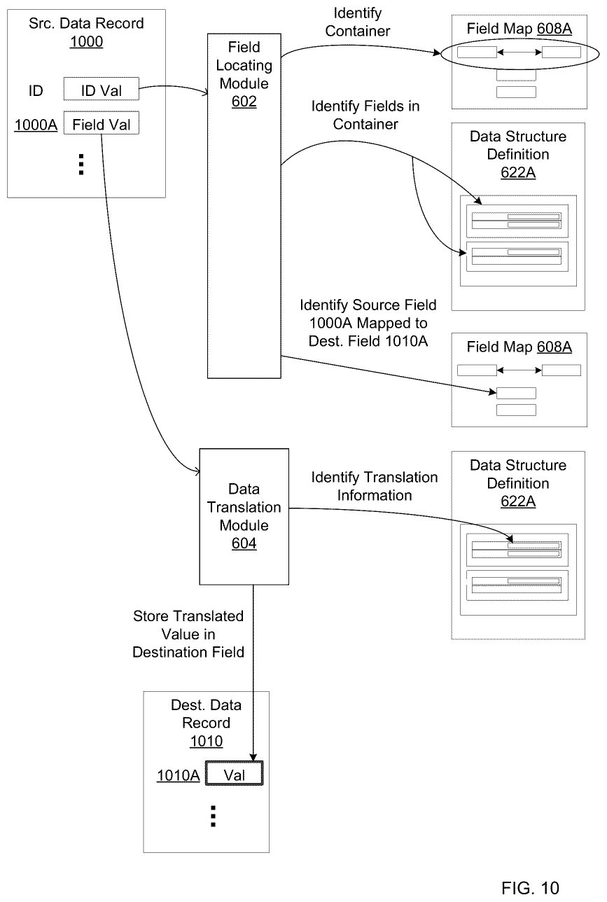

FIG. 10 illustrates transmission of data by the data transmission system 100 from a source data record 1000 (e.g., from source system 610) to a destination data record 1010 (e.g., in destination system 620), according to some embodiments of the technology described herein. As shown in FIG. 10, the transmission starts with the field locating module 602 reading the identifier of the source data record 1000 and using the identifier to identify, in the field map 608A, an associated container in the data structure definition 622A. After identifying the container, the field locating module 602 identifies the fields that are included in the container. For example, the field locating module 602 may extract destination system field identifiers from field configurations included in the container.

The field locating module 602 identifies source system fields mapped to each of the destination system fields in the container in the field map 608A. In the example of FIG. 10, the field locating module determines that source field 1000A is mapped to destination field 1010A. For example, the field locating module 602 may determine that an identifier of the destination system field 1010A is associated with an identifier of the source field 1000A in the field map 608A (e.g., in destination field mapping information associated with the destination field in the field map 608A).

As shown in FIG. 10, the data translation module 604 reads the value of source field 1000A. The data translation module 604 further identifies translation information in the data structure definition 622A associated with the destination field 1010A. The data translation module 604 uses the translation information to determine a translation of the value of source field 1000A. The data translation module 604 stores the translation in the destination field 1010A.

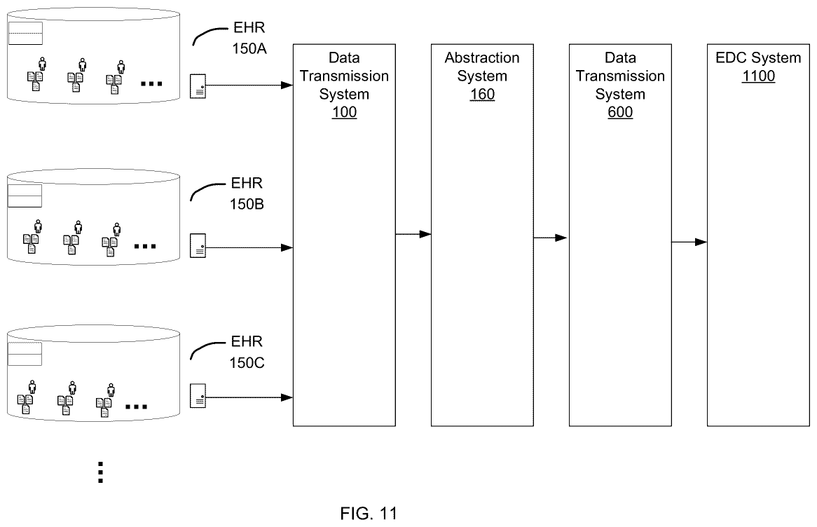

FIG. 11 is an example implementation of some embodiments of the technology described herein for transmitting data between EHR systems 150A, 150B, 150C, an abstraction system 160, and an EDC system 1100. In the example of FIG. 11, the data transmission system 100 transmits datasets from EHR systems 150A, 150B, 150C (e.g., as described herein with reference to FIG. 1B) to the abstraction system 160 (e.g., for abstraction of data points from the datasets). The data transmission system 600 transmits data from the abstraction system 160 (e.g., abstracted data) to a destination system which is the EDC system 1100. Accordingly, the data transmission systems 100, 600 may provide an automated pipeline of data between the EHR systems 150A, 150B, 150C, the abstraction system 160, and the EDC system 1100. The automated pipeline may efficiently transmit data into a data structure of the EDC system 1100 for subsequent use (e.g., in clinical trial(s), pharmaceutical research, drug development, and/or other uses).

Although in the example embodiment of FIG. 11 data transmission system 100 is depicted separately from data transmission system 600, in some embodiments, the data transmission system 100 and data transmission system 600 may be components of a single system. For example, the modules of each of the data transmission systems 100, 600 may be integrated into a single data transmission system. In some embodiments, data transmission system 100 may be separate from data transmission system 600.

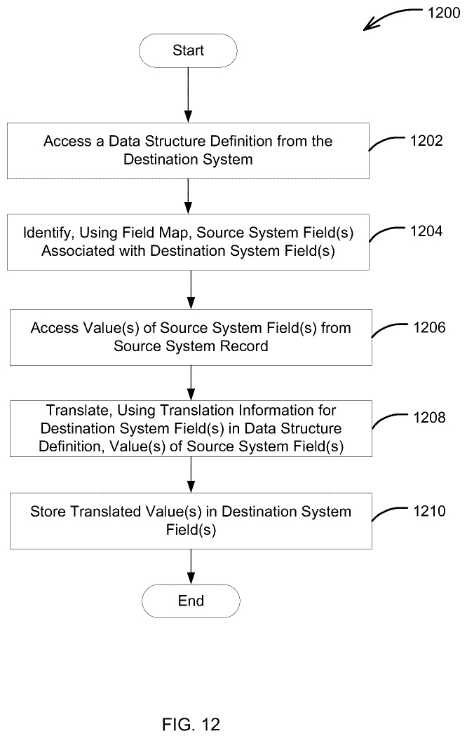

FIG. 12 is an example process 1200 for transmitting data from a source system into data records of a destination system, according to some embodiments of the technology described herein. In some embodiments, process 1200 may be performed by data transmission system 600 described herein with reference to FIG. 6. For example, process 1200 may be performed to transmit abstracted data (e.g., from an abstraction system) to data records of an EDC system. In some embodiments, the process 1200 may be performed to transmit data (e.g., abstracted data) from the source system into data records of the destination system.

Process 1200 begins at block 1202, where the system accesses a data structure definition from the destination system. The data structure definition may define a structure according to which data records of the destination system are stored. An example data structure definition is described herein with reference to FIG. 8.

Next, process 1200 proceeds to block 1204, where the system identifies, using a field map, source system field(s) from which to read value(s). In some embodiments, the system may identify the source system field(s) by: (1) identifying a container in the data structure definition associated with a source system data record including the source system field(s); (2) identify destination system field(s) in the container; and (3) identify, in the field map, source system field(s) associated with the destination system field(s) as the source system field(s) from which to read value(s). An example field map is described herein with reference to FIG. 9. The system may: (1) look up mapping information for the destination field(s) in the field map; and (2) identify source field(s) associated with the destination field(s) in the mapping information for the destination field(s).

Next, process 1200 proceeds to block 1206, where the system accesses value(s) of the identified source system field(s) from a source system record. For example, the system may access the source system data record and read the value(s) of the source system field(s) from the source system data record.

Next, process 1200 proceeds to block 1208, where the system translates, using translation information for the destination system field(s) in the data structure definition, the value(s) of the source system field(s). For example, the system may encode the value(s) of the source system field(s) into a target format of the destination system field(s). Example translations are described herein. Next, process 1200 proceeds to block 1210, where the system stores the translated value(s) in the destination system field(s).

In some embodiments, when the translated data is stored in the destination system, the destination system may use the translated data. The translated data adheres to a target format of the destination system Thus, the translated data may be used to execute queries by the destination system 620. For example, a query may use the translated data to filter data. This may allow the destination system 620 to execute data operations more efficiently.

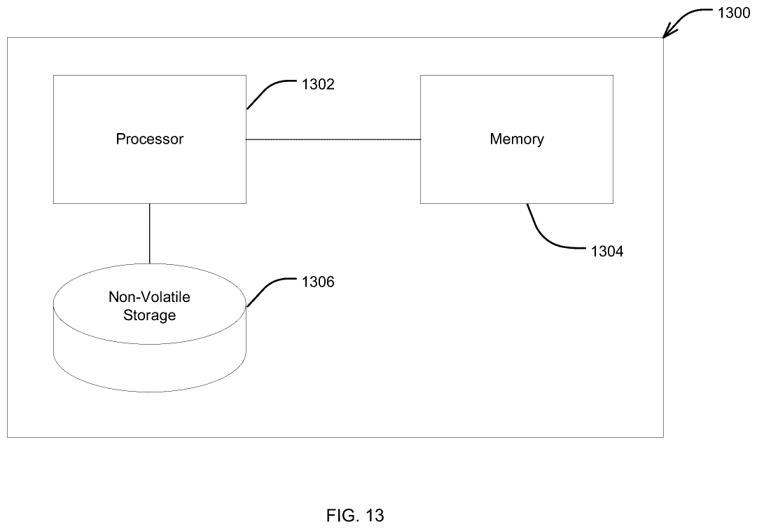

FIG. 13 shows a block diagram of an example computer system 1300 that may be used to implement some embodiments of the technology described herein. The computing device 1300 may include one or more computer hardware processors 1302 and non-transitory computer-readable storage media (e.g., memory 1304 and one or more non-volatile storage devices 1306). The processor(s) 1302 may control writing data to and reading data from (1) the memory 1304; and (2) the non-volatile storage device(s) 1306. To perform any of the functionality described herein, the processor(s) 1302 may execute one or more processor-executable instructions stored in one or more non-transitory computer-readable storage media (e.g., the memory 1304), which may serve as non-transitory computer-readable storage media storing processor-executable instructions for execution by the processor(s) 1302.

Having thus described several aspects of at least one embodiment of the technology described herein, it is to be appreciated that various alterations, modifications, and improvements will readily occur to those skilled in the art.

Such alterations, modifications, and improvements are intended to be part of this disclosure, and are intended to be within the spirit and scope of disclosure. Further, though advantages of the technology described herein are indicated, it should be appreciated that not every embodiment of the technology described herein will include every described advantage. Some embodiments may not implement any features described as advantageous herein and in some instances one or more of the described features may be implemented to achieve further embodiments. Accordingly, the foregoing description and drawings are by way of example only.

The above-described embodiments of the technology described herein can be implemented in any of numerous ways. For example, the embodiments may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. However, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, aspects of the technology described herein may be embodied as a computer readable storage medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments described above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the technology as described above. As used herein, the term “computer-readable storage medium” encompasses only a non-transitory computer-readable medium that can be considered to be a manufacture (i.e., article of manufacture) or a machine. Alternatively or additionally, aspects of the technology described herein may be embodied as a computer readable medium other than a computer-readable storage medium, such as a propagating signal.

The terms “program” or “software” are used herein in a generic sense to refer to any type of computer code or set of computer-executable instructions that can be employed to program a computer or other processor to implement various aspects of the technology as described above. Additionally, it should be appreciated that according to one aspect of this embodiment, one or more computer programs that when executed perform methods of the technology described herein need not reside on a single computer or processor, but may be distributed in a modular fashion amongst a number of different computers or processors to implement various aspects of the technology described herein.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

Various aspects of the technology described herein may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the technology described herein may be embodied as a method, of which examples are provided herein including with reference to FIGS. 3 and 7. The acts performed as part of any of the methods may be ordered in any suitable way. Accordingly, embodiments may be constructed in which acts are performed in an order different than illustrated, which may include performing some acts simultaneously, even though shown as sequential acts in illustrative embodiments.

Further, some actions are described as taken by an “actor” or a “user”. It should be appreciated that an “actor” or a “user” need not be a single individual, and that in some embodiments, actions attributable to an “actor” or a “user” may be performed by a team of individuals and/or an individual in combination with computer-assisted tools or other mechanisms.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

What is claimed is:

1. A system for ingesting data from a plurality of source systems into a destination system, the plurality of source systems storing the data in conformance with a common specification indicating a standard for transmitting datasets, the plurality of source systems including a first source system and a second source system, the system comprising:
a processor configured to execute a plurality of modules, the plurality of modules comprising:
a query generation module configured to request data from the plurality of source systems, the query generation module configured to:
generate a first query requesting a first dataset stored in the first source system, the first query including a first value of at least one field designated by the common specification for referencing a dataset;
generate a second query requesting a second dataset stored in the second source system, the second query including a second value of the at least one field designated by the common specification for referencing a dataset;
a communication module configured to communicate, through a communication network, with the plurality of source systems, the communication module configured to:
transmit, through the communication network, the first query to the first source system and the second query to the second source system; and
receive, through the communication network, after transmission of the first query and the second query:
the first dataset from the first source system; and
the second dataset from the second source system; and
a storage module configured to store, in a datastore accessible by the destination system, the first dataset and the second dataset.

2. The system of claim 1, wherein the common specification is the Fast Healthcare Interoperability Resources (FHIR) specification.

3. The system of claim 1, wherein each of at least some of the plurality of source systems is an electronic health record (EHR) system storing clinical records for a plurality of subjects.

4. The system of claim 1, wherein the at least one field designated by the specification for uniquely referencing the dataset comprises a subject identifier that uniquely identifies a subject that the dataset is associated with in a particular source system.

5. The system of claim 4, further comprising memory storing, for each of a plurality of subjects, respective values of the subject identifier used by the plurality of source systems to identify the subject, wherein:
the query generation module is configured to determine the first value of the at least one field and the second value of the at least one field by performing:
identify, in the memory, a value of the subject identifier used by the first source system to uniquely reference a first subject, wherein the first dataset is associated with the first subject in the first source system; and
identify, in the memory, a value of the subject identifier used by the second source system to uniquely reference the first subject, wherein the second dataset is associated with the first subject in the second source system.

6. The system of claim 1, wherein the first dataset comprises a first set of unstructured data stored in the first source system and the second dataset comprises a second set of unstructured data stored in the second source system.

7. The system of claim 6, wherein the first set of unstructured data comprises a first set of clinical visit note (CVN) documents associated with a first subject and the second set of unstructured data comprises a second set of CVN documents associated with a second subject.

8. The system of claim 1, wherein the processor is further configured to execute a subject identification module configured to:
identify, from among a plurality of subjects, a subject with a destination system identifier for the subject that is associated with an identifier for the subject used by the first source system; and
trigger generation of the first query to request data from the first source system after identifying the subject.

9. The system of claim 1, further comprising memory storing sets of authentication credentials for the plurality of source systems and the communication module is further configured to:
authenticate the system to the first source system using a first one of the sets of authentication credentials for the first source system to obtain a first access token;
authenticate the system to the second source system using a second one of the sets of authentication credentials for the second source system to obtain a second access token;
wherein the communication module is configured to transmit, through the communication network, the first query to the first source system and the second query to the second source system by performing:
transmit, through the communication network, the first query to the first source system using the first access token; and
transmit, through the communication network, the second query to the second source system using the second access token.

10. The system of claim 1, wherein:
the first dataset is associated with a first subject in the first source system;
the second dataset is associated with the first subject in the second source system;
determining the first value of the at least one field comprises determining a value of the at least one field used by the first source system to uniquely identify the first subject in the first source system; and
determining the second value of the at least one field comprises determining a value of the at least one field used by the second source system to uniquely identify the first subject in the second source system.

11. The system of claim 1, wherein:
the first dataset is associated with a first one of the plurality of subjects;
the second dataset is associated with a second one of the plurality of subjects;
determining the first value of the at least one field comprises determining a value of the at least one field used by the first source system to uniquely identify data associated with the first subject in the first source system; and
determining the second value of the at least one field comprises determining a value of the at least one field used by the second source system to uniquely identify data associated with the second subject in the second source system.

12. The system of claim 1, wherein the storage module is configured to store, in the datastore, the first dataset and the second dataset by performing:
read the first dataset from the first source system into a cloud-based datastore, separate from the datastore of the system;
read the second dataset from the second source system into the cloud-based datastore; and
read the first dataset and the second dataset from the cloud-based datastore into the datastore of the destination system.

13. The system of claim 1, wherein the query generation module is configured to periodically request data associated with a plurality of subjects from the plurality of source systems.

14. A method for ingesting data from a plurality of source systems into a destination system, the plurality of source systems storing the data in conformance with a common specification indicating a standard for transmitting datasets, the plurality of source systems including a first source system and a second source system, the method comprising:
using a processor to perform:
generating a first query requesting a first dataset stored in the first source system, the first query including a first value of at least one field designated by the common specification for referencing a dataset;
generating a second query requesting a second dataset stored in the second source system, the second query including a second value of the at least one field designated by the common specification for referencing a dataset;
transmitting, through the communication network, the first query to the first source system and the second query to the second source system;
receiving, through the communication network, after transmission of the first query and the second query:
the first dataset from the first source system; and
the second dataset from the second source system; and
storing, in a datastore accessible by the destination system, the first dataset and the second dataset.

15. The method of claim 14, wherein:
the at least one field designated by the specification for uniquely referencing the dataset comprises a subject identifier that uniquely identifies a subject that the dataset is associated with in a particular source system;
determining the first value of the at least one field and the second value of the at least one field comprises:
identifying a value of the subject identifier used by the first source system to uniquely reference a first subject, wherein the first dataset is associated with the first subject in the first source system; and
identifying a value of the subject identifier used by the second source system to uniquely reference the first subject, wherein the second dataset is associated with the first subject in the second source system.

16. The method of claim 14, further comprising:
identifying, from among a plurality of subjects, a subject with a destination system identifier for the subject that is associated with an identifier for the subject used by the first source system; and
triggering generation of the first query to request data from the first source system after identifying the subject.

17. The method of claim 14, wherein the datastore stores sets of authentication credentials for the plurality of source systems, and the method further comprises:
authenticating the system to the first source system using a first one of the sets of authentication credentials for the first source system to obtain a first access token; and
authenticating the system to the second source system using a second one of the sets of authentication credentials for the second source system to obtain a second access token;
wherein transmitting, through the communication network, the first query to the first source system and the second query to the second source system comprises:
transmitting, through the communication network, the first query to the first source system using the first access token; and
transmitting, through the communication network, the second query to the second source system using the second access token.

18. The method of claim 14, wherein:
the first dataset is associated with a first subject in the first source system;
the second dataset is associated with the first subject in the first source system;
determining the first value of the at least one field comprises determining a value of the at least one field used by the first source system to uniquely identify the first subject in the first source system; and
determining the second value of the at least one field comprises determining a value of the at least one field used by the second source system to uniquely identify the first user in the second source system.

19. The method of claim 14, wherein:
the first dataset is associated with a first one of the plurality of subjects;
the second dataset is associated with a second one of the plurality of subjects;
determining the first value of the at least one field comprises determining a value of the at least one field used by the first source system to uniquely identify data associated with the first subject in the first source system; and
determining the second value of the at least one field comprises determining a value of the at least one field used by the second source system to uniquely identify data associated with the second subject in the second source system.

20. A non-transitory computer-readable storage medium storing instructions that, when executed by a processor, cause the processor to perform a method for ingesting data from a plurality of source systems into a destination system, the plurality of source systems storing the data in conformance with a common specification indicating a standard for transmitting datasets, the plurality of source systems including a first source system and a second source system, the method comprising:
generating a first query requesting a first dataset stored in the first source system, the first query including a first value of at least one field designated by the common specification for referencing a dataset;
generating a second query requesting a second dataset stored in the second source system, the second query including a second value of the at least one field designated by the common specification for referencing a dataset;
transmitting, through the communication network, the first query to the first source system and the second query to the second source system;
receiving, through the communication network, after transmission of the first query and the second query:
the first dataset from the first source system; and
the second dataset from the second source system; and
storing, in a datastore accessible by the destination system, the first dataset and the second dataset.
